(19) United States
(12) Patent Application Publication
(10) Pub. No.: US 2013/0324821 A1
(43) Pub. Date: Dec. 5, 2013

(54) DEVICE FOR DIAGNOSING CONDITION OF LIVER AND METHOD OF EXAMINING CONDITION OF LIVER

(71) Applicants: Yukihiro Yokoyama, Nagoya-shi (JP); Toshio Kokuryo, Nagoya-shi (JP); Akifumi Nakagawa, Nagoya-shi (JP)

(72) Inventors: Yukihiro Yokoyama, Nagoya-shi (JP); Toshio Kokuryo, Nagoya-shi (JP); Akifumi Nakagawa, Nagoya-shi (JP)

(73) Assignee: NATIONAL UNIVERSITY CORPORATION NAGOYA UNIVERSITY, Aichi (JP)

(21) Appl. No.: 13/992,470

(22) PCT Filed: Dec. 1, 2011
(86) PCT No.: PCT/JP2011/077851

§ 371 Date: Aug. 19, 2013

(30) Foreign Application Priority Data
Dec. 10, 2010 (JP) 2010-275543

(51) Int. Cl. A61B 5/00; A61B 5/145

(52) U.S. Cl. CPC A61B 5/4244; A61B 5/14542; A61B 5/4836
USPC 600/364

(57) ABSTRACT

The diagnostic device disclosed in this application is a device for diagnosing liver condition. The diagnostic device comprises a nitric oxide sensor and an information output unit. The nitric oxide sensor can be brought into contact with hepatocytes making up the liver and detects the concentration of nitric oxide produced by the hepatocytes. The information output unit, based on the nitric oxide concentration detected by the nitric oxide sensor, outputs information for diagnosing the condition of the liver. The information output unit can, for example, output a graph showing the change over time in the nitric oxide concentration detected by the nitric oxide sensor.

### TECHNICAL FIELD

The present invention relates to a technology that may be suitably used during surgery on a liver, and relates more particularly to a technology which can monitor the condition of a liver (hepatocytes) in real time and determine the length of time for which blood flow to the liver can be stopped.

### BACKGROUND ART

When operating on the liver, to minimize bleeding, hepatectomy or the like is carried out in a state where blood flow to the liver has been stopped (ischemic state). Because there is a limit to the length of time that blood flow to the liver can be stopped, when a given length of time after stopping blood flow to the liver has elapsed, blood flow blow to the liver is restarted (reperfusion). The state of arrested blood flow to the liver (ischemic state) and the state of restarted blood flow to the liver (reperfusion state) are then repeated until the necessary procedure is completed. Surgery on the liver is thereby performed while suppressing blood flow.

### Non Patent Literature

(1) “Tolerance of the cirrhotic liver to normothermic ischemia. A clinical study of 15 patients,” by Nagasue, N., Yukaya, H., Suehiro, S. and Ogawa, Y., in Am. J. Surg. 147(6): 772-5 (June 1984)

(2) “Tolerance of the human liver to prolonged normothermic ischemia. A biological study of 20 patients submitted to extensive hepatectomy,” by Huguet, C., Nordlinger, B., Bloch, P. and Conard, J., in Arch. Surg. 113(12): 1448-51 (December 1978)

### SUMMARY OF INVENTION

When carrying out such liver ischemia/reperfusion (i.e., the repetition of an ischemic state and a reperfusion state), if blood flow to the liver is stopped for too long a period of time, hepatic ischemia/reperfusion injury will arise following surgery. On the other hand, if blood flow to the liver is stopped for too short a period of time, the liver is maintained in a good state, but the number of times ischemia/reperfusion is carried out increases. As a result, the operating time becomes longer, increasing the toll on the patient. Accordingly, the time during which blood flow to the liver is stopped can be made as long as possible within a range where hepatic ischemia/reperfusion injury does not arise. However, the length of time for which blood flow to the liver can be stopped is empirically thought to be about 15 to 25 minutes, although this differs in practice from patient to patient. This differs also with the number of times hepatic ischemia/reperfusion is carried out. Hence, decisions on the ischemic time for each patient are currently based on the experience and intuition of the physician.

One or more embodiments of the present invention provide technology capable of determining the length of time for which blood flow to the liver can be stopped.

The diagnostic device disclosed in this specification is a device for diagnosing the condition of a liver, and includes a nitric oxide (NO) sensor which can be brought into contact with hepatocytes making up the liver and detects the concentration of NO (nitric oxide) produced by the hepatocytes.

As a result of extensive research conducted by the inventors, it has been shown that NO (nitric oxide) produced in the liver is closely associated with hepatic ischemia/reperfusion injury. That is, it has become clear that placing the liver in an ischemic state increases the amount of NO (nitric oxide) produced in the liver, and that this increased amount of NO is closely associated with hepatic ischemia/reperfusion injury. In addition, it has been found that the concentration of NO (nitric oxide) produced in the liver when it has been placed in an ischemic state serves as an indicator for determining whether hepatic ischemia/reperfusion injury will arise following surgery. Because the aforementioned diagnostic device has a NO sensor for detecting the concentration of NO produced in the liver (hepatocytes), the concentration of NO produced in the liver (hepatocytes) can be monitored in real time. Therefore, the condition of the liver (hepatocytes) can be diagnosed in real time, making it possible to determine the length of time during which blood flow to the liver can be stopped.

The method of examination disclosed in this specification is a method for examining the condition of the liver, and includes the step of detecting the concentration of NO produced by hepatocytes and the step of indicating the detected NO concentration on a monitor. In this method of examination, the concentration of NO produced in the hepatocytes is disclosed on a monitor, enabling the condition of the liver to be examined in real time.

### BRIEF DESCRIPTION OF DRAWINGS

FIG. 1 is a schematic view showing, in simplified form, the construction of a NO sensor according to one or more embodiments.

FIG. 2 is a block diagram showing the configuration of a diagnostic device according to the one or more embodiments.

FIG. 3 is a flow chart showing the processing sequence of a diagnostic device during surgery on the liver.

FIG. 4 is a first graph showing an example of the change over time in the NO concentration when hepatic ischemia/reperfusion was carried out in mice.

FIG. 5 is a second graph showing an example of the change over time in the NO concentration when hepatic ischemia/reperfusion was carried out in mice.

FIG. 6 is a graph showing the relationship between the number of ischemia/reperfusion cycles and the amount of NO produced by placing the liver in an ischemic state.

FIG. 7 is a diagram which visually shows the amount of NO produced by placing the liver in an ischemic state.

### DETAILED DESCRIPTION OF INVENTION

The diagnostic device disclosed in this specification may further includes an information output unit which, based on the NO concentration detected by the NO sensor, outputs information for diagnosing the condition of the liver. This arrangement enables a physician or the like to diagnose the condition of the liver based on the information output by the information output unit.

Here, the information output unit may, for example, output a graph showing the change over time in the NO concentration detected by the NO sensor. This arrangement enables the change over time in NO concentration (i.e., the change over time in the condition of the liver) to be understood at a glance.

The information output unit may, at least when the liver has been placed in an ischemic state, output a value obtained by integrating on a time axis the NO concentration detected by the NO sensor. Integrating on a time axis the NO concentration detected by the NO sensor gives the amount of NO produced (NO production) in the liver (hepatocytes). According to research by the inventors, NO production in the liver has been shown to have a negative correlation with liver damage. That is, it has been shown that if the extent of liver damage is high, the amount of NO produced in the liver when the liver was placed in an ischemic state is low. Hence, when the value obtained by integrating the NO concentration on a time axis is output, a physician can objectively determine the extent of liver damage based on the value that was output.

The information output unit may, at least when the liver has been placed in an ischemic state, output a value obtained by integrating on a time axis the value obtained by subtracting the NO concentration detected just before ischemia is started from the NO concentration detected by the NO sensor. With this arrangement, because the value obtained by subtracting the NO concentration detected just before ischemia is started from the NO concentration detected by the NO sensor is integrated on a time axis, the amount of NO produced when the liver has been placed in an ischemic state is output. Hence, the influence on the liver of having been placed in an ischemic state can be satisfactorily diagnosed.

The information output unit may, at least when the liver has been placed in an ischemic state, output a value obtained by differentiating on a time axis the NO concentration detected by the NO sensor. By differentiating the NO concentration on a time axis, the percent change over time in the NO concentration can be obtained. It is possible in this way as well to satisfactorily diagnose the condition of the liver.

Also, the diagnostic device disclosed in this specification may further include a first determination unit which determines timing for switching from an ischemic state to a reperfusion state based on at least one of: the NO concentration detected by the NO sensor when the liver has entered an ischemic state; the value obtained by integrating on a time axis the value obtained by subtracting the NO concentration detected just before ischemia is started from the NO concentration detected by the NO sensor when the liver has entered an ischemic state; and the differential value of the NO concentration detected by the NO sensor when the liver has entered an ischemic state. This arrangement makes it possible to switch from an ischemic state to a reperfusion state under suitable timing.

In this arrangement, the first determination unit may determine the timing for switching from an ischemic state to a reperfusion state to be when the differential value of the NO concentration detected by the NO sensor becomes equal to or less than a first preset value.

In addition, the diagnostic device disclosed herein may further include a second determination unit which determines timing for switching from a reperfusion state to an ischemic state based on at least one of the NO concentration detected by the NO sensor when the liver has entered a reperfusion state from an ischemic state; and the differential value of the NO concentration detected by the NO sensor when the liver has entered a reperfusion state from an ischemic state. This arrangement makes it possible to switch from a reperfusion state to an ischemic state under suitable timing.

In this arrangement, the second determination unit may determine the timing for switching from a reperfusion state to an ischemic state to be when the absolute value of the differential value of the NO concentration detected by the NO sensor becomes equal to or less than a second preset value.

Also, the second determination unit may determine the timing for switching from a reperfusion state to an ischemic state to be when the NO concentration detected by the NO sensor becomes equal to or less than a third preset value and the absolute value of the differential value of the NO concentration detected by the NO sensor becomes equal to or less than a fourth preset value.

The technology disclosed in this specification can output useful information utilizing the NO concentration Cᵢ detected by the NO sensor at the time that the liver, when repeatedly switched between an ischemic state and a reperfusion state, is switched from an i^{th} ischemic state to an i^{th} reperfusion state. For example, in one embodiment, the diagnostic device may further include a first annunciator which indicates that, at the time that the liver is switched from the i^{th} ischemic state to the i^{th} reperfusion state, the i^{th} reperfusion state time should be made longer than the i−1^{th} reperfusion state time when the concentration value difference Cᵢ₋₁-C₁ is equal to or greater than a fifth preset value. Alternatively, the diagnostic device may further include a second annunciator which indicates that, at the time that the liver is switched from the i^{th} ischemic state to the i^{th} reperfusion state, the i+1^{th} ischemic state time should be made shorter than the i^{th} ischemic state time when the concentration value difference Cᵢ₋₁-Cᵢ is equal to or greater than a sixth preset value.

Or the technology disclosed in this specification may output useful information utilizing the value Sᵢ obtained by integrating on a time axis, when the liver is repeatedly switched between an ischemic state and a reperfusion state, the value obtained by subtracting the NO concentration detected just before the i^{th} ischemia is started from the NO concentration detected by the NO sensor in the i^{th} ischemic state. For example, in yet another embodiment, the diagnostic device may further include a third annunciator which indicates that, at the time that the liver is switched from the i^{th} ischemic state to the i^{th} reperfusion state, the i^{th}reperfusion state time should be made longer than the i−1^{th} reperfusion state time when the integrated value difference Sᵢ₋₁-Sᵢ is equal to or greater than a seventh preset value. Alternatively, the diagnostic device may further include a fourth annunciator which indicates that, at the time that the liver is switched from the i^{th} ischemic state to the i^{th} reperfusion state, the i+1^{th} ischemic state time should be made shorter than the i^{th} ischemic state time when the integrated value difference Sᵢ₋₁-Sᵢ is equal to or greater than an eighth preset value. (EMBODIMENTS) The diagnostic device of the present embodiment may be used when operating on a liver (more specifically, at the time of liver ischemia/reperfusion in liver surgery), and is preferably used for diagnosing the length of time for which blood flow to the liver can be stopped. As shown in FIG. 2, the diagnostic device of this embodiment comprises a NO sensor 10, an arithmetic unit 24 and a monitor 32.

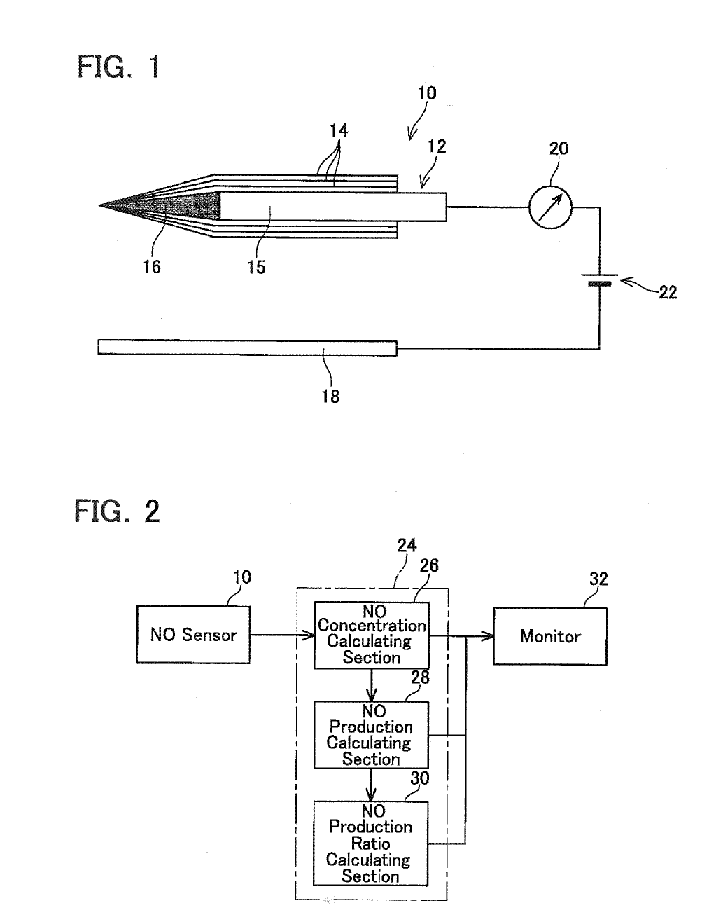

The NO sensor 10 detects the concentration of NO produced by the liver (hepatocytes). As shown in FIG. 1, the NO sensor 10 has a working electrode 12 and a reference electrode 18. The working electrode 12 has a base portion 15 and a sensor portion 16 provided at the end of the base portion 15. The base portion 15 is formed of silver or platinum (e.g., platinum-iridium). The sensor portion 16 is formed of carbon. The base portion 15 and the sensor portion 16 are covered by a NO-selective membrane 14 which is permeable to NO (nitric oxide). The NO-selective membrane 14 is formed of three types of polymeric membranes, and covers the sidewalls of the sensor portion 16 and the base portion 15. Covering the end and sides of the working electrode 12 with the NO-selective membrane 14 enhances the durability and measurement sensitivity of the NO sensor 10. The reference electrode 18 is formed of carbon.

In order to detect, using the NO sensor 10, the concentration of NO produced by the liver, the base portion 15 of the working electrode 12 is connected to the positive pole of a power supply 22 through an ammeter 20, and the reference electrode 18 is connected to the negative pole of the power supply 22. Next, the working electrode 12 and the reference electrode 18 are brought into contact with the liver (such as by sticking electrodes 12 and 18 into the liver). Upon applying the potential of the power supply 22 in this manner between the working electrode 12 and the reference electrode 18, a current corresponding to the amount of NO produced by the liver flows between the working electrode 12 and the reference electrode 18. This current is measured with the ammeter 20, and the concentration of NO produced in the liver from this measured current is calculated.

Use may be made of, for example, the Nitric Oxide Sensor available from World Precision Instruments as the NO sensor 10. However, the NO sensor 10 is not limited to the foregoing Nitric Oxide Sensor; other known NO sensors may be used.

The arithmetic unit 24 is connected to the NO sensor 10 (specifically, to the ammeter 20 of the NO sensor 10). Current values measured with the ammeter 20 are input to the arithmetic unit 24. The arithmetic unit 24 carries out various types of processing using the current values input from the NO sensor 10. The arithmetic unit 24 may comprise a computer or processor having a CPU, ROM and RAM, and a memory which stores a program. The arithmetic unit 50 executes the program stored in the memory, functioning as a NO concentration calculating section 26, a NO production calculating section 28 and a NO production ratio calculating section 30. In addition, an input unit (not shown) is connected to the arithmetic unit 24, and the start and end of hepatic ischemia/reperfusion are input to the arithmetic unit 24.

The NO concentration calculating section 26 uses the current value measured with the ammeter 20 of the NO sensor 10 to calculate by voltammetry the concentration of NO produced in the liver. That is, the power supply 22 connected to the NO sensor 10 is programmed so as to vary over time in a predetermined pattern the potential applied between the working electrode 12 and the reference electrode 18 during NO measurement. The NO concentration calculating section 26 acquires a current-time characteristic from the current value measured with the ammeter 20 and calculates the NO concentration from this current-time characteristic.

The NO production calculating section 28 calculates, from the NO concentration calculated by the NO concentration calculating section 26, the amount of NO produced in the liver (NO production) in the period during which the liver is in an ischemic state. Specifically, the NO production calculating section 28 integrates on a time axis the value obtained by subtracting the NO concentration calculated by the NO concentration calculating section 26 just before the liver is placed in an ischemic state from the NO concentration calculated by the NO concentration calculating section 26 when the liver is in an ischemic state, and calculates the production of NO produced by the liver in the period during which the liver is in an ischemic state. Because the NO production calculating section 28 integrates the value obtained by subtracting the NO concentration detected just before the liver is placed in an ischemic state from the NO concentration detected by the NO sensor 10, it calculates the amount of NO that has increased (NO production) as a result of placing the liver in an ischemic state.

The NO production ratio calculating section 30 computes the ratio, at the end of a second or subsequent ischemic state, between the amount of NO produced when the liver was in the second or subsequent ischemic state and the amount of NO produced when the liver was in a first ischemic state. The amount of NO produced in the liver when it has been placed in an ischemic state is higher when the condition of the liver is good, and is lower when the condition of the liver is poor. Hence, a smaller value for (NO production during second or subsequent ischemic state)/(NO production during first ischemic state) is presumed to indicate a larger degree of liver damage. Accordingly, by calculating such ratios with the NO production ratio calculating section 30, the condition of the liver can be appropriately diagnosed.

A monitor 32 is connected to the arithmetic unit 24. The monitor 32 displays various types of data, such as the NO concentration calculated with the arithmetic unit 24, a graph showing the change over time in this NO concentration, and the NO production.

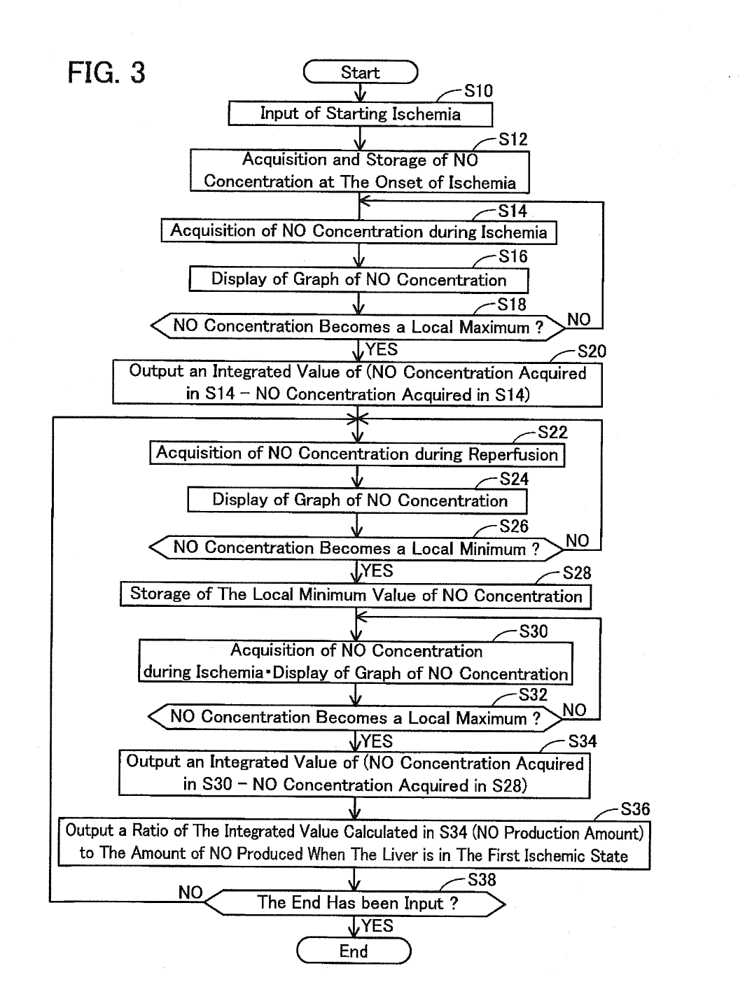

Next, the sequence for monitoring the condition of the liver using the above-described diagnostic device will be described in conjunction with FIG. 3. In order to monitor the condition of the liver, first the working electrode 12 and the reference electrode 18 are set in the liver to be monitored (the patient's liver). Next, blood flow to the liver is stopped, thereby starting ischemia, concurrent with which “onset of ischemia” is input to the diagnostic device (S10). When “onset of ischemia” is input, the arithmetic unit 24 acquires the NO concentration at the onset of ischemia, and the NO concentration thus acquired is saved (S12). Specifically, the power supply 22 varies in a predetermined pattern the voltage applied between the working electrode 12 and the reference electrode 18, and the current value observed at that time is measured with the ammeter 20. The arithmetic unit 24 acquires a current-time characteristic from the current value measured by the ammeter 20, calculates the NO concentration from this current-time characteristic, and stores the calculated NO concentration in a memory (not shown).

Next, when a predetermined time elapses after the completion of processing in Step S12 (i.e., upon the arrival of the detection cycle by the NO sensor 10), measurement of the NO concentration is carried out with the NO sensor 10 (S14). Measurement of the NO concentration is carried out in the same way as in Step S12. That is, the power supply 22 applies a voltage in a predetermined pattern between the working electrode 12 and the reference electrode 18, and the arithmetic unit 24 calculates the NO concentration from the current value measured with the ammeter 20.

Next, the arithmetic unit 24 outputs to the monitor 32 a graph showing, from the NO concentration calculated in Step S14, the change over time in the NO concentration detected by the NO sensor 10 (S16). The change over time in NO concentration is thus displayed on the monitor 32, making it possible for a physician or the like to diagnose in real time the condition of the liver from the NO concentration displayed on the monitor 32.

Next, the arithmetic unit 24 determines, from the NO concentration calculated in Step S14, whether the NO concentration detected in NO sensor 10 is a maximum value (S18). Specifically, the arithmetic unit 24 determines whether the NO concentration detected in the present detection cycle is lower than the NO concentration detected in the immediately preceding detection cycle. If the NO concentration detected in the present detection cycle is lower than the NO concentration detected in the immediately preceding detection cycle, it is determined that the NO concentration detected by the NO sensor 10 was a maximum value in the immediately preceding detection cycle. On the other hand, if the NO concentration detected in the present detection cycle is higher than the NO concentration detected in the immediately preceding detection cycle, it is determined that the NO concentration detected by the NO sensor 10 was not a maximum value. Processing in Step S18 is used to determine whether to switch from an ischemic state to a reperfusion state. At the time of ischemia/reperfusion, the ischemic state and the reperfusion state are alternately repeated. In the ischemic state, the NO concentration detected by the NO sensor 10 rises, and in the reperfusion state, the NO concentration detected by the NO sensor 10 falls. As a result, by determining whether the NO concentration detected by the NO sensor 10 was a maximum value, it can be determined whether the liver has switched from an ischemic state to a reperfusion state.

In cases where the NO concentration detected by the NO sensor 10 has not become a maximum value (“NO” in Step S18), the sequence returns to Step S14 and processing from Step S14 is repeated. In each predetermined detection cycle, the NO concentration detected by the NO sensor 10 is calculated, and a graph of the calculated NO concentration is displayed on the monitor 32.

If it has been determined that the NO concentration detected by the NO sensor 10 is a maximum value (“YES” in Step S18), the arithmetic unit 24 integrates on a time axis the value obtained by subtracting the NO concentration saved in Step S12 from the NO concentration calculated in Step S14, and the amount of NO produced in the liver while in an ischemic state is calculated (S20). Specifically, for each of the NO concentrations calculated in Step S14 during the interval from when ischemia was started in Step S10 until ischemia was determined to have ended in Step S18, the NO concentration saved in Step S12 is subtracted, the detection cycle (time) is multiplied with the numerical value obtained by subtraction, and the NO production is calculated by summating the results. The calculated NO production is output to the monitor 32 and displayed on the monitor 32. In Step S18, because the NO concentration obtained in the immediately preceding detection cycle is determined to be a maximum value, integration processing in Step S20 is carried out using the value up to the NO concentration obtained in the immediately preceding detection cycle.

Moving on to Step S22, the arithmetic unit 24 carries out measurement of the NO concentration with the NO sensor 10. Measurement of the NO concentration is carried out in the same way as in Steps S12 and S14, giving the concentration of NO produced in the liver during reperfusion.

Next, as in Step S16, the arithmetic unit 24 outputs to the monitor 32 a graph showing the change over time in the NO concentration detected by the NO sensor 10 (S24). As a result, the change over time in NO concentration during reperfusion is displayed on the monitor 32.

Next, the arithmetic unit 24 determines, from the NO concentration computed in Step S22, whether the NO concentration detected by the NO sensor 10 is a minimum value (S26). Specifically, the arithmetic unit 24 determines, from the NO concentration detected in the immediately preceding detection cycle, whether the NO concentration detected in the present detection cycle is higher. If the NO concentration detected in the present detection cycle is higher than the NO concentration detected in the immediately preceding detection cycle, the NO concentration detected with the NO sensor 10 is determined to have been a minimum value in the immediately preceding detection cycle. On the other hand, if the NO concentration detected in the present detection cycle is lower than the NO concentration detected in the immediately preceding detection cycle, the NO concentration detected with the NO sensor 10 is determined to not have been a minimum value. By way of the processing in this Step S26, it is determined whether the liver was switched from a reperfusion state to an ischemic state. As noted above, the NO concentration detected by the NO sensor 10 falls when the liver is in a reperfusion state, and the NO concentration detected by the NO sensor 10 rises when the liver is an ischemic state. Hence, whether the liver has been switched from a reperfusion state to an ischemic state is determined by determining whether the NO concentration detected by the NO sensor 10 is a minimum value.

If the NO concentration detected by the NO sensor 10 is determined to not be a minimum value (“NO” in Step S26), the sequence returns to Step S22 and processing from Step S22 is repeated. As a result, for each given detection cycle, the NO concentration detected by the NO sensor 10 is calculated, and a graph of this calculated NO concentration is displayed on the monitor 32.

If the NO concentration detected by the NO sensor 10 is determined to be a minimum value (“YES” in Step S26), the arithmetic unit 24 saves the minimum value of the NO concentration in the memory (S28). Specifically, the NO concentration detected in the immediately preceding detection cycle, which is a detection cycle for which the NO concentration was determined to be a minimum value, is saved as a minimum value.

Next, the arithmetic unit 24, as in Steps S14 and S16, acquires the NO concentration during ischemia using the NO sensor 10, and displays on the monitor 32 a graph showing the change over time in the NO concentration detected with the NO sensor 10 (S30). Then, the arithmetic unit 24, in order to determine whether the liver has been switched from an ischemic state to a reperfusion state, determines whether the NO concentration detected by the NO sensor 10 is a maximum value (S32). Processing in Step S32 is carried out in the same way as processing in above Step S18.

If the NO concentration detected by the NO sensor 10 has been determined to not be a maximum value (“NO” in Step S32), the sequence returns to Step S30 and processing from Step S30 is repeated. On the other hand, if the NO concentration detected by the NO sensor 10 has been determined to be a maximum value (“YES” in Step S32), the arithmetic unit 24 integrates on a time axis the value obtained by subtracting the NO concentration saved in Step S28 from the NO concentration calculated in Step S30, and calculates the amount of NO produced while the liver is in an ischemic state (S34). Specifically, in the interval from when it is determined in Step S26 that ischemia has restarted until it is determined in Step S32 that ischemia has ended, for each NO concentration calculated in Step S30, the NO concentration saved in Step S28 is subtracted therefrom, and the NO production is calculated by integrating the results. The calculated NO production is output to and displayed on the monitor 32.

Next, the arithmetic unit 24 calculates the ratio of the amount of NO produced in Step S34 to the amount of NO produced when the liver is in the first ischemic state (the NO production calculated in Step S20), and outputs this calculated value to the monitor 32 (S36). As described above, at a smaller value for (NO production calculated in Step S34)/(NO production calculated in Step S20), the degree of liver damage is larger. Therefore, if the value of the ratio calculated in Step S36 and shown on the monitor 32 is small, the physician can determine that the degree of liver damage is large. This in turn makes it possible for the physician to take a suitable measure (that is, the measure of shortening the ischemia time or lengthening the reperfusion time).

Moving on to Step S38, the arithmetic unit 24 determines whether the end of ischemia/reperfusion has been input (S38). If the end of ischemia/reperfusion has not been input (“NO” in Step S38), the sequence returns to Step S22 and processing from Step S22 is repeated. As a result, monitoring of the liver continues. On the other hand, if the end of ischemia/reperfusion has been input (“YES” in Step S38), the arithmetic unit 24 brings processing to an end.

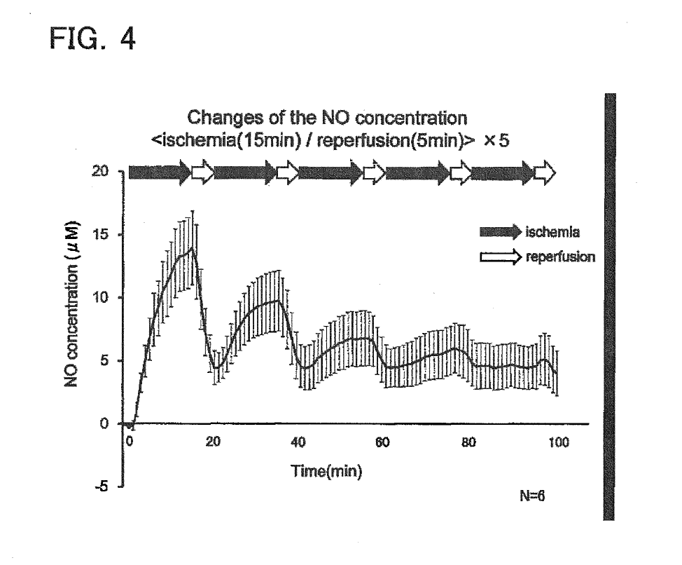

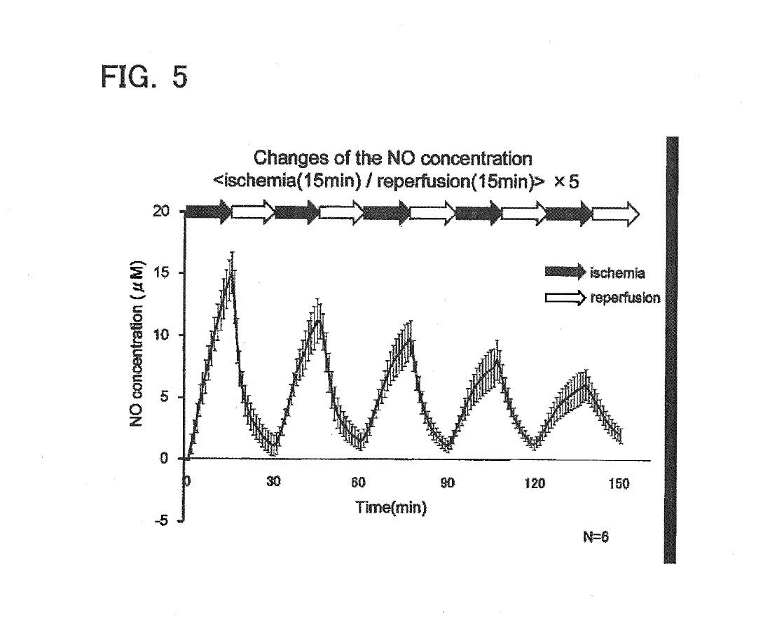

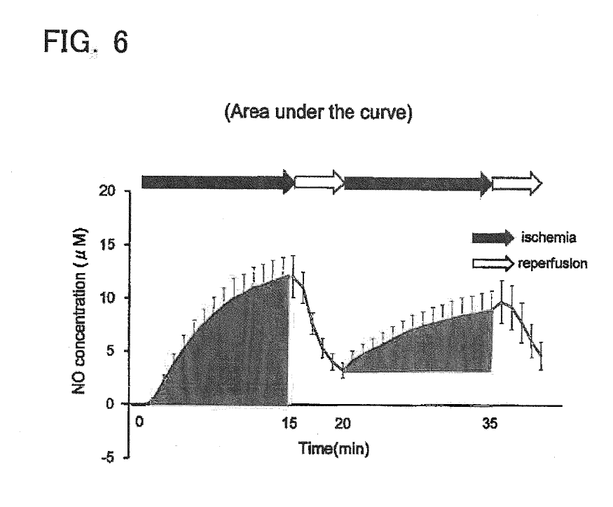

Examples are described below in which ischemia/reperfusion was carried out on the livers of mice, and the mouse livers were monitored using the above diagnostic device. FIGS. 4 and 5 are graphs showing the change over time in the NO concentration detected with the NO sensor 10. FIG. 4 is a graph for cases in which the ischemia time was set to 15 minutes and the reperfusion time was set to 5 minutes, and FIG. 5 is a graph for cases in which the ischemia time was set to 15 minutes and the reperfusion time was set to 15 minutes. As is evident from FIGS. 4 and 5, when the livers were placed in an ischemic state, the NO concentration rose, and when the livers were placed in a reperfusion state, the NO concentration fell. It is apparent from a comparison of FIGS. 4 and 5 that when the reperfusion time is short, the NO concentration does not fall sufficiently in the reperfusion stop times (the second and subsequent ischemia start times), and the NO concentration peak values at the second and subsequent ischemia end times also become low. Presumably, this is because liver function does not recover sufficiently when the reperfusion time is short, leading to an increase in the degree of liver damage. On the other hand, when the reperfusion time is long, the NO concentration decreases sufficiently at the reperfusion stop times (the second and subsequent ischemia start times) in addition to which the NO concentration peak values at the second and subsequent ischemia end times increase. Accordingly, the higher the amount of NO produced in the liver during ischemia, the more likely it is for good liver function to be maintained and for a determination that liver damage is unlikely to occur to be made.

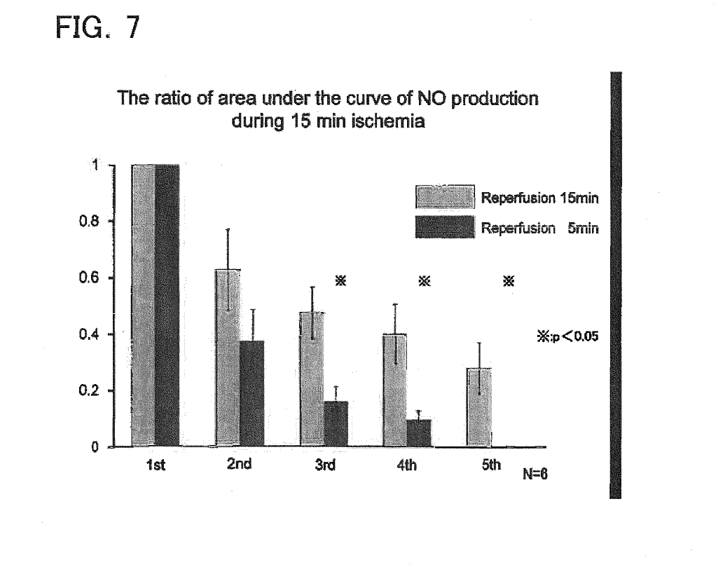

FIG. 6 is a bar graph showing the results obtained from computing the amount of NO produced during the ischemic interval in each ischemic state. In the diagram, the light-colored columns represent examples in which the reperfusion time was set to 15 minutes (the examples shown in FIG. 5), and the dark-colored columns represent examples in which the reperfusion time was set to 5 minutes (the examples shown in FIG. 4). Also, the NO production was computed by integrating on a time axis the values obtained by subtracting the NO concentration immediately before starting ischemia from the NO concentration detected by the NO sensor 10 (that is, the computed NO production was the solid gray region shown in FIG. 7). As is evident from FIG. 6, the NO production decreased as the number of ischemic states increased. In particular, when reperfusion was short (5 minutes), the degree of decline in the NO production was larger. Moreover, mice in which the reperfusion time was set to 5 minutes had higher AST and ALT (indicators of liver damage) values than mice in which the reperfusion time was set to 15 minutes. Accordingly, it was found that the amount of NO produced by the liver during the ischemic intervals can be used as an indicator for evaluating liver damage.

As is apparent from the above explanation, with the diagnostic device according to this embodiment, the concentration of NO produced in the liver can be monitored in real time by using the NO sensor 10. The condition of the liver (degree of liver damage) can thus be evaluated in real time, making it possible to determine suitable ischemia and reperfusion times. As a result, it is possible to length the ischemia time and shorten the operating time while preventing post-operative liver damage.

Moreover, the above diagnostic device outputs to the monitor 32 not only the NO concentration during hepatic ischemia/reperfusion, but also the amount of NO produced by the liver in the interval during which it is placed in an ischemic state and the ratio of such NO production. Therefore, because the degree of liver damage has been quantified and can be objectively assessed, it is possible to suitably carry out a determination of the ischemia time.

In the foregoing embodiment, the amount of NO produced by the liver during the ischemic period was determined by integrating on a time axis the value obtained by subtracting the NO concentration detected by the NO sensor 10 just before ischemia begins from the NO concentration detected by the NO sensor 10. However, the present invention is not limited to this mode; for example, the NO production may be calculated by directly integrating the NO concentration detected with the NO sensor 10. Even when the NO production is thus calculated, the degree of liver damage can be satisfactorily determined.

Also, in the foregoing embodiment, the degree of liver damage was qualitatively assessed based on NO production in the ischemic period, although various parameters may be used as indicators for assessing the degree of liver damage. For example, the slope of the NO concentration curve obtained by differentiating on a time axis the NO concentration detected by the NO sensor 10 when the liver has been placed in an ischemic state may be used as the indicator. As shown in the experimental results described above, the shorter the reperfusion time (the larger the degree of liver damage), the lower the NO concentration peak value and the more gradual the slope of the NO concentration. Hence, the degree of liver damage can be determined by using the slope of the NO concentration as the indicator. For example, it is possible to use the following as the slope of the NO concentration: {(NO concentration at end of ischemia (i.e., peak value of NO concentration))−(NO concentration at onset of ischemia)}/ischemia time. Alternatively, the NO concentration at the end of ischemia (NO concentration peak value) may be used as the indicator. Yet another possibility is to use the ratio of the NO concentration slope (or NO concentration peak value) to the NO concentration slope in the first ischemic state (or the peak value of the NO concentration) as the indicator of liver damage.

In the diagnostic device of the above-described embodiment, by having the arithmetic unit 24 execute a predetermined program, the arithmetic unit 24 may be made to function as a first determination unit which determines timing for switching from an ischemic state to a reperfusion state based on at least one of: the NO concentration detected by the NO sensor 10 when the liver has entered an ischemic state; the value obtained by integrating on a time axis the value obtained by subtracting the NO concentration detected just before ischemia is started from the NO concentration detected by the NO sensor 10 when the liver has entered an ischemic state; and the differential value of the NO concentration detected by the NO sensor 10 when the liver has entered an ischemic state. When the first determination unit determines the timing for switching from an ischemic state to a reperfusion state, information to this effect may be indicated on the monitor 32, or it may be indicated with an audible sound. Because the operator is thus made aware of the timing for switching from the ischemic state to the reperfusion state, switching to a reperfusion state can be carried out with appropriate timing.

The first determination unit may be configured so as to determine the timing for switching from an ischemic state to a reperfusion state to be when the differential value of the NO concentration detected by the NO sensor becomes equal to or less than a first preset value. The differential value of the NO concentration may be used as the indicator for predicting the NO producing ability of the liver (state of liver activity). That is, when the differential value of the NO concentration is large, it can be determined that the liver will probably maintain thereafter a good state of activity. On the other hand, when the NO concentration differential value is small, it can be determined that the liver will probably be unable to maintain a good state of activity. Hence, when the differential value of the NO concentration becomes equal to or less than a first preset value, by switching from an ischemic state to a reperfusion state, injury to the liver can be prevented beforehand from arising.

Also, in the above-described diagnostic device, by having the arithmetic unit 24 execute a predetermined program, the arithmetic unit 24 may be made to function as a second determination unit which determines timing for switching from a reperfusion state to an ischemic state based on at least one of: the NO concentration detected by the NO sensor 10 when the liver has entered a reperfusion state from an ischemic state; and the differential value of the NO concentration detected by the NO sensor 10 when the liver has entered a reperfusion state from an ischemic state. Here, when the second determination unit determines the timing for switching from a reperfusion state to an ischemic state, information to this effect may be indicated on the monitor 32, or it may be indicated with an audible sound. Because the operator is thus made aware of the timing for switching from the reperfusion state to the ischemic state, switching to an ischemic state can be carried out with appropriate timing.

The second determination unit may determine the timing for switching from a reperfusion state to an ischemic state to be when the absolute value of the differential value of the NO concentration detected by the NO sensor 10 becomes equal to or less than a second preset value. As is apparent from a comparison of FIGS. 4 and 5, when the condition of the liver becomes good due to the reperfusion states, the absolute value of the differential value of the NO concentration becomes smaller. Therefore, by using the absolute value of the differential value of the NO concentration, the timing for switching from a reperfusion state to an ischemic state can be suitably determined. Also, the second determination unit may determine the timing for switching from a reperfusion state to an ischemic state to be when the NO concentration detected by the NO sensor 10 is equal to or less than a third preset value and the absolute value of the differential value of the NO concentration detected by the NO sensor 10 is equal to or less than a fourth preset value. By also taking into consideration the NO concentration, the timing for switching from a reperfusion state to an ischemic state can be more appropriately determined.

Also, by utilizing the NO concentration C, detected by the NO sensor 10 at the time that the liver, when repeatedly switched between an ischemic state and a reperfusion state, is switched from an i^{th} (i being a positive integer) ischemic state to an i^{th} reperfusion state, it is possible to determine the subsequent reperfusion state time or ischemic state time. For example, the diagnostic device may additionally have a first annunciator which indicates that, at the time that the liver is switched from the i^{th} ischemic state to the i^{th} reperfusion state, the i^{th} reperfusion state time should be made longer than the i−1^{th} reperfusion state time when the concentration value difference Cᵢ₋₁-Cᵢ is equal to or greater than a fifth preset value. If the concentration value difference Cᵢ₋₁-Cᵢ is large, that much more of a strain can be determined to be acting on the liver. Hence, when the concentration value difference Cᵢ₋₁-Cᵢ is large, injury to the liver can be prevented from occurring by lengthening the reperfusion state time. In cases where this arrangement is employed, the monitor 32 can be used as the first annunciator. Also, the diagnostic device may additionally have a second annunciator which indicates that, at the time that the liver is switched from the i^{th} ischemic state to the i^{th} reperfusion state, the i+1^{th} ischemic state time should be made shorter than the i^{th} ischemic state time when the concentration value difference Cᵢ₋₁-Cᵢ is equal to or greater than a sixth preset value. If the concentration value difference Cᵢ₋₁-Cᵢ is large, a large strain acts on the liver. Hence, injury to the liver can be prevented from occurring by shortening the subsequent ischemic state time. The monitor 32 can be made to function as the second annunciator.

By utilizing the value Sᵢ obtained by integrating on a time axis, when the liver is repeatedly switched between an ischemic state and a reperfusion state, the value obtained by subtracting the NO concentration detected just before the i^{th} ischemia is started from the NO concentration detected by the NO sensor in the i^{th} ischemic state, it is possible to determine the subsequent reperfusion state time and ischemic state time. For example, the diagnostic device may additionally have a third annunciator which indicates that, at the time that the liver is switched from the i^{th} ischemic state to the i^{th} reperfusion state, the i^{th} reperfusion state time should be made longer than the i−1^{th} reperfusion state time when the integrated value difference Sᵢ₋₁-Sᵢ is equal to or greater than a seventh preset value. At a large integrated value difference Sᵢ₋₁-Sᵢ that much more of a strain can be determined to be acting on the liver. Hence, when the integrated value difference Sᵢ₋₁-Sᵢ is large, injury to the liver can be prevented from occurring by lengthening the i^{th} reperfusion state time. In cases where this arrangement is employed, the monitor 32 can be used as the third annunciator. Also, the diagnostic device may additionally have a fourth annunciator which indicates that, at the time that the liver is switched from the i^{th} ischemic state to the i^{th} reperfusion state, the i+1^{th} ischemic state time should be made shorter than the i^{th} ischemic state time when the integrated value difference Sᵢ₋₁-Sᵢ is equal to or greater than an eighth preset value. When the integrated value difference Sᵢ₋₁-Sᵢ is large, injury to the liver can be prevented from occurring by shortening the subsequent ischemic state time. The monitor 32 can be made to function as the fourth annunciator.

The embodiments of the present invention are described above in detail, but these examples are merely illustrative and place no limitation on the scope of the patent claims. The technology described in the patent claims also encompasses various changes and modifications to the specific examples described above.

Furthermore, it is to be understood that the technical elements described in the present specification and the drawings exhibit technical usefulness solely or in various combinations thereof and shall not be limited to the combinations described in the claims at the time of filing. The embodiments illustrated in the present specification and the drawings may achieve a plurality of objectives at the same time, and technical usefulness is exhibited by attaining any one of such objectives.

1.-16. (canceled)

17. A device for diagnosing the condition of a liver, the device comprising a nitric oxide sensor configured to be brought into contact with hepatocytes making up the liver, the nitric oxide sensor being configured to detect the concentration of nitric oxide produced by the hepatocytes.

18. The diagnostic device according to claim 17, further comprising an information output unit, based on the nitric oxide concentration detected by the nitric oxide sensor, is configured to output information for diagnosing the condition of the liver.

19. The diagnostic device according to claim 18, wherein the information output unit is configured to output a graph showing a change over time in the nitric oxide concentration detected by the nitric oxide sensor.

20. The diagnostic device according to claim 19, wherein the information output unit, at least when the liver has been placed in an ischemic state, is configured to output a value obtained by integrating on a time axis the nitric oxide concentration detected by the nitric oxide sensor.

21. The diagnostic device according to claim 20, wherein the information output unit, at least when the liver has been placed in an ischemic state, is configured to output a value obtained by integrating on a time axis the value obtained by subtracting the nitric oxide concentration detected just before ischemia is started from the nitric oxide concentration detected by the NO sensor.

22. The diagnostic device according to claim 21, wherein the information output unit, at least when the liver has been placed in an ischemic state, is configured to output a value obtained by differentiating on a time axis the nitric oxide concentration detected by the nitric oxide sensor.

23. The diagnostic device according to claim 22, further comprising a first determination unit being configured to determine timing for switching from an ischemic state to a reperfusion state based on at least one of: the nitric oxide concentration detected by the nitric oxide sensor when the liver has entered an ischemic state; the value obtained by integrating on a time axis the value obtained by subtracting the nitric oxide concentration detected just before ischemia is started from the nitric oxide concentration detected by the nitric oxide sensor when the liver has entered an ischemic state; and the differential value of the nitric oxide concentration detected by the nitric oxide sensor when the liver has entered an ischemic state.

24. The diagnostic device according to claim 23, wherein the first determination unit is configured to determine the timing for switching from an ischemic state to a reperfusion state to be when the differential value of the nitric oxide concentration detected by the nitric oxide sensor becomes equal to or less than a first preset value.

25. The diagnostic device according to claim 23, further comprising a second determination unit being configured to determine timing for switching from a reperfusion state to an ischemic state based on at least one of: the nitric oxide concentration detected by the nitric oxide sensor when the liver has entered a reperfusion state from an ischemic state; and the differential value of the nitric oxide concentration detected by the nitric oxide sensor when the liver has entered a reperfusion state from an ischemic state.

26. The diagnostic device according to claim 25, wherein the second determination unit is configured to determine the timing for switching from a reperfusion state to an ischemic state to be when the absolute value of the differential value of the nitric oxide concentration detected by the nitric oxide sensor becomes equal to or less than a second preset value.

27. The diagnostic device according to claim 25, wherein the second determination unit is configured to determine the timing for switching from a reperfusion state to an ischemic state to be when the nitric oxide concentration detected by the nitric oxide sensor becomes equal to or less than a third preset value and the absolute value of the differential value of the nitric oxide concentration detected by the nitric oxide sensor becomes equal to or less than a fourth preset value.

28. The diagnostic device according to claim 27, wherein Cᵢ is the nitric oxide concentration detected by the nitric oxide sensor at the time that the liver is switched from an i^{th} ischemic state to an i^{th} reperfusion state, in a case where the liver is repeatedly switched between an ischemic state and a reperfusion state, and
the diagnostic device further comprises a first annunciator being configured to indicate that the i^{th} reperfusion state time should be made longer than the i−1^{th} reperfusion state time when a concentration value difference Cᵢ₋₁-Cᵢ is equal to or greater than a fifth preset value.

29. The diagnostic device according to claim 28, further comprising a second annunciator being configured to indicate that the i+1^{th} ischemic state time should be made shorter than the i^{th} ischemic state time when a concentration value difference Cᵢ₋₁-Cᵢ is equal to or greater than a sixth preset value.

30. The diagnostic device according to claim 29, wherein Sᵢ is the value obtained by integrating on a time axis the value obtained by subtracting the nitric oxide concentration detected just before the i^{th} ischemia is started from the nitric oxide concentration detected by the nitric oxide sensor in the i^{th} ischemic state, in a case where the liver is repeatedly switched between an ischemic state and a reperfusion state, and
the diagnostic device further comprises a third annunciator being configured to indicate that the i^{th} reperfusion state time should be made longer than the i−1^{th} reperfusion state time when an integrated value difference Sᵢ₋₁-Sᵢ is equal to or greater than a seventh preset value.

31. The diagnostic device according to claim 30, further comprising a fourth annunciator being configured to indicate that the i+1^{th} ischemic state time should be made shorter than the i^{th} ischemic state time when an integrated value difference Sᵢ₋₁-Sᵢ is equal to or greater than an eighth preset value.

32. A method for examining the condition of a liver, the method comprising:
detecting the concentration of nitric oxide produced by hepatocytes; and
indicating the detected nitric oxide concentration on a monitor.

33. A device for diagnosing the condition of a liver, the device comprising:
a nitric oxide sensor configured to detect the concentration of nitric oxide produced by the liver; and
a monitor in communication with the nitric oxide sensor, the monitor being configured to display a graph showing a change over time in the nitric oxide concentration detected by the nitric oxide sensor.

34. The diagnostic device according to claim 33, further comprising a processor in communication with the nitric oxide sensor and the monitor, the processor being configured to calculate a value for diagnosing the condition of the liver, and wherein the monitor is configured to display the calculated value.

35. The diagnostic device according to claim 34, wherein the processor is further configured to determine, based upon the value for diagnosing the condition of the liver, timing for switching from an ischemic state to a reperfusion state.

36. The diagnostic device according to claim 35, wherein the processor is further configured to determine, based upon the value for diagnosing the condition of the liver, timing for switching from a reperfusion state to an ischemic state.
